(19) United States
(12) Patent Application Publication
(10) Pub. No.: US 2011/0066066 A1
(43) Pub. Date: Mar. 17, 2011

(54) DEVICE FOR THE DETECTION OF SYMPTOMATIC JAW BEHAVIOUR

(75) Inventors: Paul Van Kemenade, Schijndel (NL); Joost Verdonk, 's-hertogenbosch (NL)

(73) Assignee: BRUXTEC B.V., Uden (NL)

(21) Appl. No.: 12/677,360

(22) PCT Filed: Sep. 10, 2008
(86) PCT No.: PCT/NL08/50595

§ 371 Date: Nov. 17, 2010

(30) Foreign Application Priority Data
Sep. 10, 2007 (NL) 2000850

(51) Int. Cl. A61B 5/0488

(52) U.S. Cl. 600/546

(57) ABSTRACT

A device for detecting symptomatic jaw behaviour in a user includes a mouthpiece (1) to be worn in a mouth of the user. The mouthpiece is provided with at least one sensor (2) for electronically receiving a jaw muscle signal. The at least one sensor is herein coupled to an electronic processing element (10) with a clock element (11) and with an electronic storage element (12) for the purpose of detecting a recorded muscle signal in time and storing it in optionally processed form.

The present invention relates to a device for detecting symptomatic jaw behaviour in a user, comprising a mouthpiece which is adapted and intended to be worn in a mouth of the user, which mouthpiece is provided with at least one sensor for electronically receiving a jaw muscle signal.

The invention relates particularly here to a device for indicating different forms of so-called bruxism at an early stage, so that an appropriate course of treatment can be adapted thereto in good time. Bruxism is an involuntary or subconscious para-function which takes place during the day as well as at night, and is manifested in a static or dynamic contact between the chewing surface complexes of the lower and upper jaw, wherein a pronounced neuromuscular activity occurs. These contacts do not form part of the normal functional physiology. In respect of bruxism a distinction is made between two kinds of symptomatic jaw behaviour, i.e. clenching of the teeth on the one hand and grinding of the teeth on the other. In addition, other forms of jaw behaviour occur which may or may not be related.

A device specifically aiming to reduce the above stated forms of bruxism is known from European patent application EP 1.706.078 of applicant. This known device comprises a sensor for receiving neural jaw muscles signals as indication of probably occurring bruxism. Above a determined, optionally user-specific threshold value this results in a correction signal in the form of an electrostatic pulse which is generated to the jaw in order to end the bruxism.

This known device provides per se an effective means against bruxism. The known device is nevertheless not always found to be equally effective for all patients. This is manifested for instance in correcting pulses occurring when there is no bruxism or, conversely, such a pulse not occurring when the user is experiencing bruxism.

The present invention has for its object, among others, to provide a device which is able to respond more effectively to the individual user.

In order to achieve the stated object a device of the type stated in the preamble has the feature according to the invention that the at least one sensor is coupled to electronic processing means with clock means and with electronic storage means for the purpose of detecting a recorded muscle signal in time and storing it in optionally processed form. The invention is based here on the insight that bruxism can occur in different forms and degrees, in addition to other forms of possibly symptomatic jaw behaviour. If such jaw behaviour is perceived as irksome or harmful, and must therefore be alleviated, it is of great importance to precisely determine the exact nature of the behaviour at the earliest possible stage in order to be able to adapt an appropriate course of treatment thereto. As a result of the invention an advance or interim specific diagnosis can thus be prepared per user by recording and storing for later analysis his/her specific jaw behaviour for a period of time.

The recorded muscle signals can here be stored in raw form or be preprocessed and/or filtered. In both forms the invention provides a device which records and stores jaw behaviour for a longer period of time. On the basis of the thus collected data a reliable diagnosis can be made in respect of the nature of the detected phenomena. If desired, a specific treatment can then be adapted thereto. It is expected that the device according to the invention will be accepted easily and quickly by a user, so that the device according to the invention will already provide sufficient insight into the nature of the jaw malfunction before irreparable damage has occurred to particularly his/her teeth, and a treatment can be initiated in good time.

Although the processing means can per se be applied optionally together with the storage means externally, i.e. separately of the mouthpiece, a particular embodiment of the device according to the invention has the feature that the mouthpiece comprises the processing means and the electronic storage means. The device is thus integrated in exceptionally compact manner in the mouthpiece, wherein individual, user-specific parameters can also be processed semi-permanently or permanently in the device.

In order to limit a necessary storage capacity of the storage means a further particular embodiment of the device according to the invention has the feature that the processing means comprise a programmable microprocessor which at least during operation is able and adapted, on the basis of a program code which can be programmed therein, to diagnose a recorded jaw muscle signal and to store it in diagnosed form. Because the processing means thus perform an analysis of the raw muscle signals and store only the result of this analysis, a significant gain in information density can be achieved and, in particular, no memory space need be allocated to a non-useful part of the received signal from which no, or hardly any, useful information can be retrieved.

In a further particular embodiment the device according to the invention herein has the feature that the processing means are able and adapted on the basis of the program code to at least discriminate between jaw muscle signals representative of specific jaw behaviour taken from a group comprising clenching of teeth and grinding of teeth for the purpose of storing these as events in time. The device is thus able for instance to store the intensity and degree of regularity of occurrence of at least these two symptoms in time for the purpose of a later evaluation by a medical practitioner. This latter is able to assess on the basis thereof whether and which treatment is then indicated.

In order to limit power consumption in the mouthpiece and for a practically unlimited processing capacity of the detected signals, a further particular embodiment of the device according to the invention has the feature that at least the storage means are integrated into the mouthpiece and can be coupled, optionally in wired manner, to processing means located outside the mouthpiece for the purpose of electronic data exchange therewith. The external processing means can for instance here comprise a personal computer loaded with appropriate analysis software.

For the purpose of an electric power supply to electronic components accommodated in the mouthpiece, a further particular embodiment of the device according to the invention has the feature that the mouthpiece comprises electronic power supply means. Such a power source can for instance comprise an optionally rechargeable (button cell) battery. A first further particular embodiment has the feature however that the power supply means comprise at least one electrochemical cell which is able and adapted to co-act with saliva in the mouth of the user for the purpose of generating an electric supply voltage therefrom, while a second further particular embodiment is characterized in that the power supply means comprise at least one electromagnetic coupling member which is able and adapted to intercept and convert an externally generated electromagnetic alternating field into an electronic supply voltage. In the first case a supply voltage is derived from an electrochemical potential between electrodes which are adapted for this purpose and which enter into contact with the saliva of the user, while in the second case the supply voltage is provided externally and transmitted to the device wirelessly by electromagnetic means. In both cases the power supply means autonomously provide a continuous supply voltage without having to be periodically replaced or (re)charged. Alternatively use can also be made of the kinetics of the jaw joints for the purpose of deriving a supply voltage therefrom, for instance by piezoelectric means or in other manner.

In order to avoid malfunction of the device in the unlikely event the memory capacity of the storage means reaches a maximum and to always have available the most recent data, a further particular embodiment of the device according to the invention has the feature that the storage means are organized as an electronically readable FIFO (First In First Out) slide register. If the storage means become full, the oldest data will herein automatically make way to provide space for the most recent data. Use is preferably made here of a memory capacity which is sufficiently large to cover a period of time sufficiently long to allow reliable conclusions to be drawn from the stored data.

In a further particular embodiment the device according to the invention has the feature that the processing means are able and adapted to sample received muscle signals at a sampling frequency, in particular one in the order of about 1 kHz, and to store the signals in digitized form. Such a digitized storage not only simplifies further electronic processing and analysis of the thus stored signals, but also reduces the storage capacity necessary therefor. The sampling frequency applied here always involves a balance between storage capacity and energy consumption on the one hand and resolution or precision on the other.

In a further particular embodiment the device according to the invention has the feature that the at least one sensor extends from the mouthpiece and is able and adapted to co-act with at least one jaw element of the user for the purpose of fixing the mouthpiece in the mouth. The sensors thus have a dual function, wherein the fixation on or between one or more jaw elements also ensures a close and fixed contact between the sensor and the jaw (muscles).

A further embodiment of the device according to the invention has the feature that the mouthpiece comprises at least one electrode which can be coupled to a device for administering an electrical stimulus, and more particularly that the at least one sensor also forms the at least one electrode. The device can thus be employed not only diagnostically but also therapeutically.

The invention will now be further elucidated on the basis of an exemplary embodiment and an associated drawing. In the drawing:

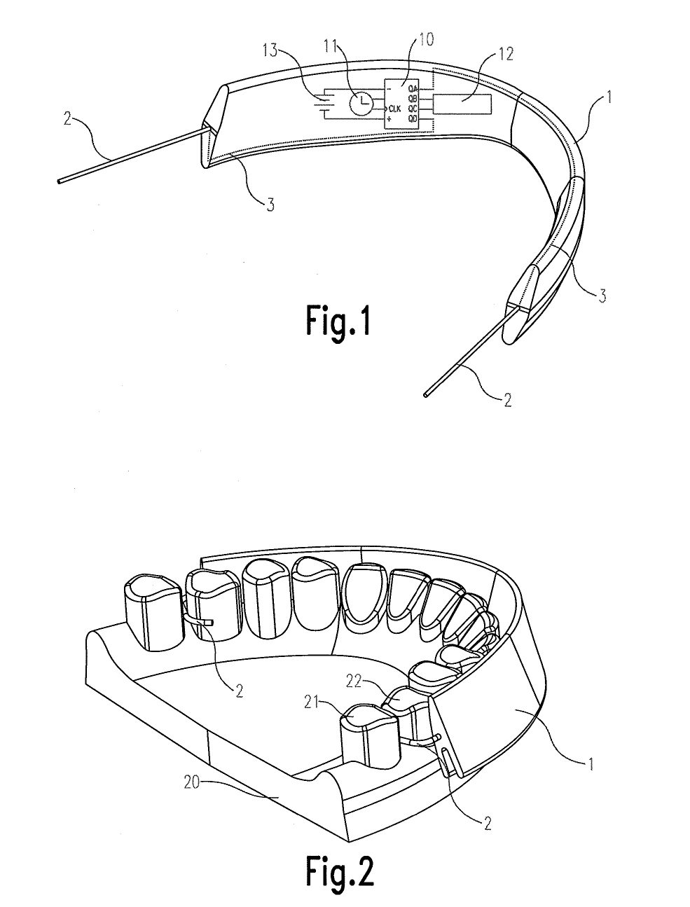

FIG. 1 is a perspective view of an exemplary embodiment of the device according to the invention;

FIG. 2 shows the device of FIG. 1 in mounted position;

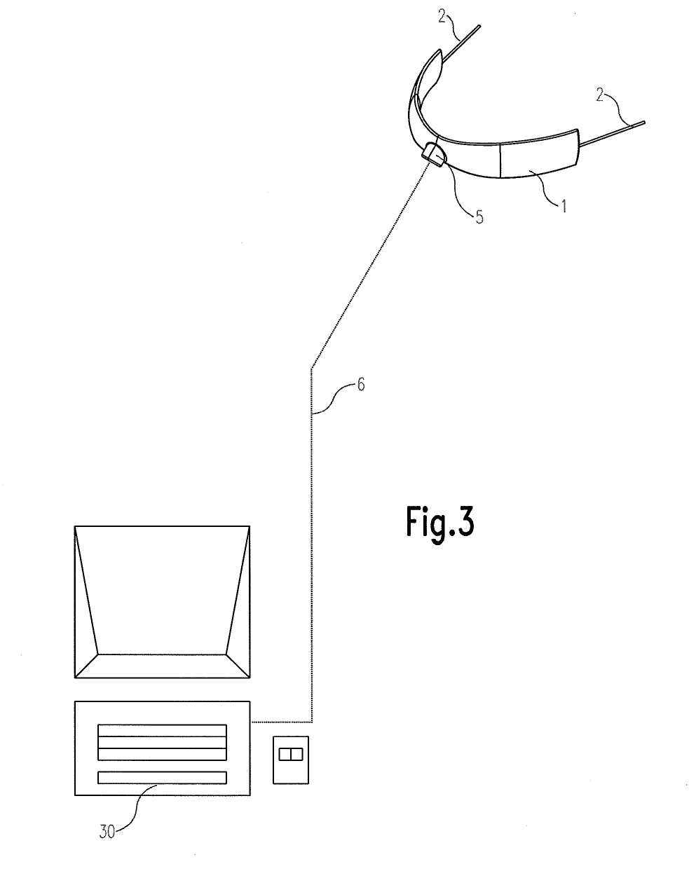

FIG. 3 shows a front view and partly schematic representation of the device of FIG. 1; and

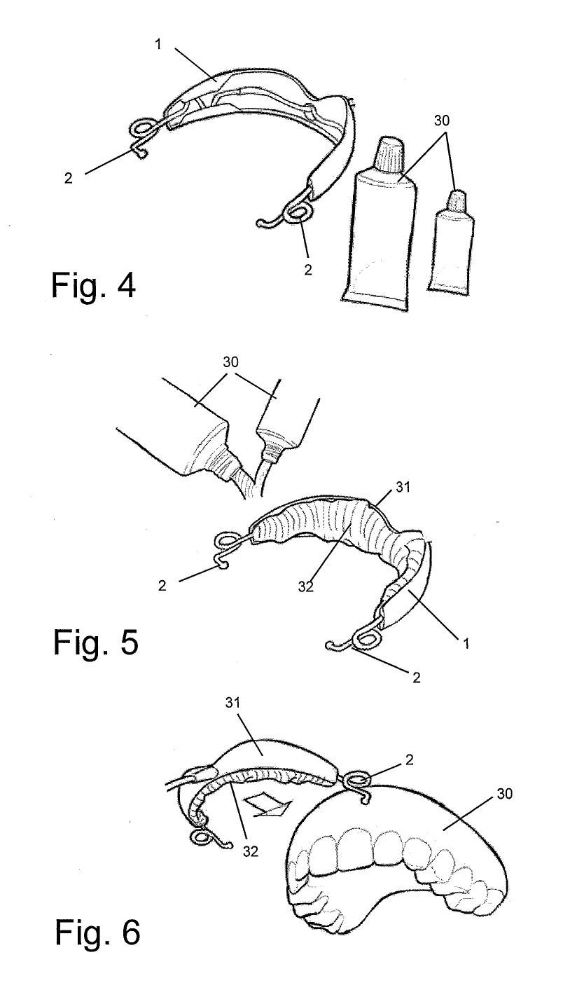

FIGS. 4-9 show successive stages of fitting of a device according to the invention.

The figures are otherwise purely schematic and not drawn to scale. Some dimensions in particular may be exaggerated to greater or lesser extent for the sake of clarity. Corresponding parts are designated as far as possible in the figures with the same reference numeral.

An exemplary embodiment of a device according to the invention is shown in FIG. 1. The device comprises a mouthpiece 1 intended to be worn in the mouth of the user during a night's sleep and/or in some cases even during the day. The mouthpiece can be manufactured from a thermoplastic plastic and can then be deformed to some extent to the shape of the jaw by the user at increased temperature, for instance in hot water, but can also be fitted specifically to size by a dentist or a dental technician from a standard orthodontic material. A close fit will in that case already guarantee sufficient fixation of the mouthpiece in the mouth against a part of for instance a lower jaw of the user.

Situated on the mouthpiece is a set of antennas which serve as sensor for the purpose of receiving neural electromagnetic signals representative of a momentary activity of the chewing muscles. The received electronic signals are exchanged via a set of schematically shown conductor tracks 3 in the mouthpiece having a processing unit 10 integrated therein. In this example sensors 2 comprise a set of metal probes which extend from mouthpiece 1 and allow deformation with for instance pliers or other tool for the purpose of hooking round or between one or more jaw elements 21,22 as shown in FIG. 2. Fixation of mouthpiece 1 with sensors 2 in the mouth is thus in any case guaranteed, this being important for the integrity and consistency of the signals to be picked up with sensors 2.

According to the invention processing unit 10 is coupled to clock means 11 and an electronically writable and readable memory device 12. In this example the memory and clock means are shown separately of processing means 10, although it will be apparent that with present-day semiconductor technology some or even all of these parts can also be accommodated in a shared integrated circuit, so that the mouthpiece will comprise only one alien component.

Power supply means 13 provide electric power supply to electronic components 10-12. These can be formed by an optionally rechargeable battery, wherein use is advantageously made, for the purpose of compactness, of a flat button cell format. Instead or in addition, the power supply means can however also comprise an antenna with which an externally generated electromagnetic alternating field is intercepted and converted into a suitable supply voltage, or use is made of a wet chemical galvanic cell of a set of electrodes which, in combination with the saliva of the user, brings about an electrochemical potential for power supply to components 10-13. These two latter options provide the advantage that components 10-13 do not require a replaceable battery for their power supply and can be cast or otherwise incorporated in hermetically moisture-tight manner in the material of the mouthpiece.

The device is inserted by a user before a night's sleep and during the night then continuously records jaw muscle activity derived from the electro-muscular (EMG) signals from the chewing muscles received with the sensors. By here applying a relatively high sampling frequency of about at least 1 kHz, the device is able to store the detected EMG signal with a high accuracy within the relevant frequency range. Substantially no useful information is thus lost. If desired, the mouthpiece can also be worn during the day so as to also obtain insight into the jaw behaviour of the user in the waking state.

In principle the device according to the invention detects all EMG signals above a sensitivity threshold; not specifically of the chewing muscles but of all activated muscles. Different muscle groups have signals with a peak in their own specific frequency range. Many muscles, such as those of the arms and neck, have a peak in a frequency range lying below 100 Hz. The chewing muscles have a maximum which is slightly higher, i.e. between about 90 and 150 Hz. By filtering on this basis the device according to the invention can differentiate and detect specific symptomatic muscle behaviour and be optimized for detection of signals from chewing muscles above other muscles. This increases the diagnostic power of the device. One consequence is for instance that the device can be designed such that it produces little or no signal when the user for instance turns the head or moves an arm but, conversely, produces much signal when the jaws are placed or clamped onto each other. This also makes it possible to place the sensors some distance from the muscles to be measured.

Supported by the clock means, these signals are stored in time in memory 12 of the device and thus stored at least temporarily. Use can be made here of storage of the raw data as they are directly received, although in order to save memory space the processing unit 10 comprises in this example a microprocessor which, loaded with suitable software, is able to carry out an analysis of the received signals and to store only an intensity and incidence of symptomatic jaw behaviour specifically recognized thereby. These data can later be read for interpretation by an expert, optionally supported by additional software.

Memory 12 is accessed by the processing unit as a FIFO (First In First Out) slide register so that the oldest data will always make room for the most recent in the unlikely event the memory capacity should prove insufficient for the storage of all data. This achieves that the above stated interpretation is based on the most recent measurement data.

For a further processing of the stored data the memory 12 of the device can be read regularly or after a period of time. For this purpose the device has on a front side thereof a connector 5 with which a cable connection 6 can be effected to further processing means 30, see FIG. 3. The further processing means comprise here a personal computer 30 which has sufficient fixed memory capacity to store the data over a sufficiently long period for the purpose of further evaluation.

The PC further has appropriate analysis software to support an expert for the purpose of drawing from the stored data reliable conclusions on which an effective course of treatment can then be based. In the case of a battery-powered device the connector 5 can optionally be used simultaneously to supply a charging current, and the device can moreover be adapted such that programming of the processing unit is possible via the connector. In this latter case processing algorithms and threshold values appropriate to a user can be written into the device.

In the case of specific symptomatic jaw muscle behaviour, such as for instance bruxism, a diagnosis that has been made will indicate a treatment targeted at this behaviour. The diagnosis device according to the invention can be employed per se for the purpose of administering correcting stimuli in the mouth of the user as described in the literature as effective treatment for bruxism. For this purpose the mouthpiece can further be equipped with electrodes, or even the sensors can be employed as electrodes. In such a case use is preferably made of a wired or wireable embodiment of the device so that sufficient electric power is available for administering appropriate correction signals. In such a case the electrodes are coupled in or on a biofeedback system in order to generate an appropriate electrical stimulus to the jaw when a jaw malfunction to be corrected is detected, so that the jaw relaxes again and an opperant conditioning eventually occurs which wholly suppresses or at least reduces the jaw malfunction to acceptable proportions.

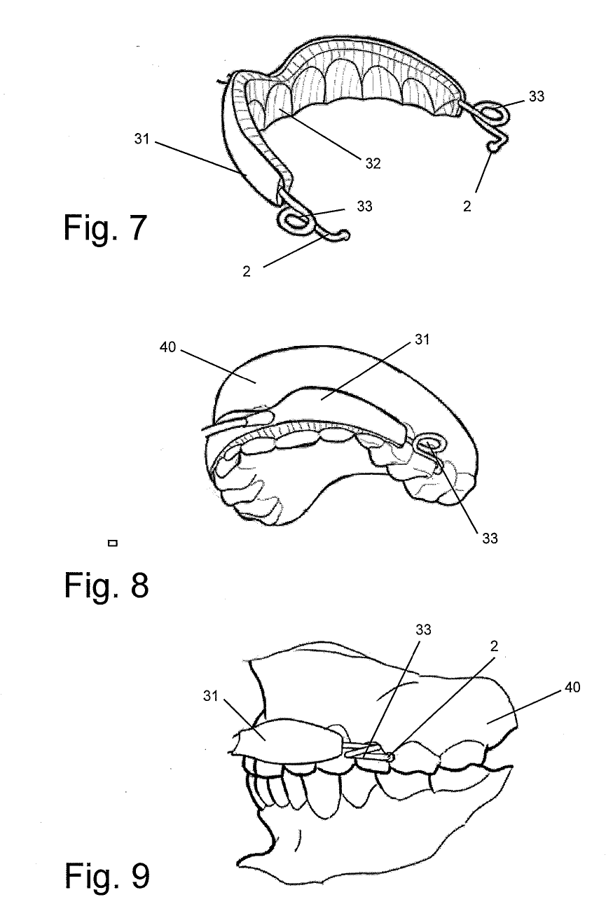

For a close fitting onto a set of teeth of a user the device according to the invention can be precisely made to measure therefor. An embodiment of a procedure to be followed for this purpose is shown in successive stages in FIG. 7 and further. The mouthpiece of the device that must be placed in the mouth comprises in this case an outer shell 31 having therein the processing means, clock means and peripheral memory as has been described above together with an electronic power source and a data/power supply connector or wireless interface (not shown). In addition, this part comprises among other components two sensors 2 extending on either side thereof. Outer shell 13 is manufactured from a relatively form-retaining biocompatible plastic. Use is advantageously made here of a limited number of standard sizes which approximate the size of the jaw of the user.

For fitting on the jaw 40 of the user, the mouthpiece comprises an inner shell 32, see FIG. 5, which is arranged to the size of the upper teeth 40 of the user using a curable two-component paste 30. For this purpose the paste 30 is applied in a liberal quantity in outer shell 31, see FIG. 5, and pressed firmly against the upper teeth 30 of the user, see FIG. 6. The viscous paste will herein set precisely in accordance with the shape of the teeth of the user.

After being removed and cured, an outer shell 32 thus results, see FIG. 10, which connects at least substantially close-fittingly onto the teeth of the user. This precise fitting provides a close fit whereby mouthpiece 31,32 remains firmly fixed on the upper jaw 40 of the user without further anchoring means being required, see FIG. 8. Instead of a two-component paste, use can also be made of similar other materials for this purpose, particularly a thermally permanently deformable thermoplastic and other deformable materials, which cure under the influence of optionally visible light, radiation or in other manner so as to take on a permanent form.

Sensors 2 are otherwise formed and oriented in this example such that they lie accurately at the correct location against jaw 40 of the user when mouthpiece 31,32 is fitted onto teeth 40, see FIG. 9. Suitable electro-muscular signals with a sufficiently high signal-noise ratio are reproducibly detected at this location. A resilient turn 33 in sensors 2 herein provides extra spring pressure at the intended location and thereby enhances a mechanical contact at that position.

Although the invention has been further elucidated above with reference to only a single exemplary embodiment, it will be apparent that the present invention is in no way limited thereto. On the contrary, many variations and embodiments are still possible for a person with ordinary skill in the art without departing from the scope of the invention.

The collected data can thus optionally be used as parameters for the purpose of precisely adapting a therapy, optionally supported with a correcting device, to a user at a later stage. A user can for instance be envisaged here who always only falls asleep after 60 minutes instead of after another more or less standard period of time. In this case a therapeutic treatment can be initiated correspondingly later.

It is further possible to generate, on the basis of the measured signal, a signal which indicates whether there is bruxism activity or another disorder, such as for instance apnoea, and using this signal to activate a camera/video recording or to wake the patient in order to thus avoid a potentially threatening situation.

Use is made in the exemplary embodiment of a cable connection to external processing means for reading and/or programming the device. Use can however also be made instead of a wireless coupling on the basis of an optionally standardized protocol for data transfer, such as for instance Bluetooth, WiFi and the like.

1. Device for detecting symptomatic jaw behaviour in a user, comprising a mouthpiece which is adapted and intended to be worn in a mouth of the user, which mouthpiece is provided with at least one sensor for electronically receiving a jaw muscle signal, characterized in that the at least one sensor is coupled to electronic processing means and with electronic storage means for the purpose of detecting a recorded muscle signal during a time span and storing it in optionally processed form in time.

2. Device as claimed in claim 1, characterized in that the mouthpiece comprises the processing means and the electronic storage means.

3. Device as claimed in claim 1, characterized in that the processing means comprise a programmable microprocessor which at least during operation is able and adapted, on the basis of a program code which can be programmed therein, to diagnose a recorded jaw muscle signal and to store it in diagnosed form.

4. Device as claimed in claim 3, characterized in that the processing means are able and adapted on the basis of the program code to at least discriminate between jaw muscle signals representative of specific jaw behaviour taken from a group comprising clenching of teeth and grinding of teeth for the purpose of storing these as events in time.

5. Device as claimed in claim 4, characterized in that at least the storage means are integrated into the mouthpiece and can be coupled, optionally in wired manner, to processing means located outside the mouthpiece for the purpose of electronic data exchange therewith.

6. Device as claimed in claim 1, characterized in that the mouthpiece comprises electronic power supply means.

7. Device as claimed in claim 6, characterized in that the power supply means comprise at least one electrochemical cell which is able and adapted to co-act with saliva in the mouth of the user for the purpose of generating an electric supply voltage therefrom.

8. Device as claimed in claim 6, characterized in that the power supply means comprise at least one electromagnetic coupling member which is able and adapted to intercept and convert an externally generated electromagnetic alternating field into an electronic supply voltage.

9. Device as claimed in claim 1, characterized in that the storage means are organized as an electronically readable FIFO (First In First Out) slide register.

10. Device as claimed in claim 1, characterized in that the at least one sensor extends from the mouthpiece and is able and adapted to co-act with at least one jaw element of the user for the purpose of fixing the mouthpiece in the mouth.

11. Device as claimed in claim 1, characterized in that the processing means are able and adapted to sample received muscle signals at a sampling frequency, in particular one in the order of about 1 kHz, and to store the signals in digitized form.

12. Device as claimed in claim 1, characterized in that the mouthpiece comprises at least one electrode which can be coupled to a device for administering an electrical stimulus.

13. Device as claimed in claim 12, characterized in that the at least one sensor also forms the at least one electrode.

14. Device as claimed in claim 2, characterized in that the processing means comprise a programmable microprocessor which at least during operation is able and adapted, on the basis of a program code which can be programmed therein, to diagnose a recorded jaw muscle signal and to store it in diagnosed form.
